(19) United States
(12) Patent Application Publication
(10) Pub. No.: US 2025/0032053 A1
(43) Pub. Date: Jan. 30, 2025

(54) SYSTEM AND METHOD FOR ENHANCING USER EXPERIENCE OF AN ELECTRONIC DEVICE DURING ABNORMAL SENSATION

(71) Applicant: Samsung Electronics Co., Ltd., Suwon-si (KR)

(72) Inventors: Choice CHOUDHARY, Tohana (IN); Desh Deepak AGARWAL, Noida (IN); Mangal SINGH, Chandigarh (IN); Ankit AGARWAL, Ghaziabad (IN); Sobita CHOUDHARY, Haryana (IN)

(21) Appl. No.: 18/912,010

(22) Filed: Oct. 10, 2024

Related U.S. Application Data
(63) Continuation of application No. PCT/KR2023/003895, filed on Mar. 23, 2023.

(30) Foreign Application Priority Data
Jun. 17, 2022 (KR) 202211034851

(51) Int. Cl. A61B 5/00; A61B 5/0205; A61B 5/11; A61B 5/117; G16H 20/00

(52) U.S. Cl. CPC A61B 5/7282; A61B 5/0205; A61B 5/1116; A61B 5/117; A61B 5/7203; G16H 20/00

(57) ABSTRACT

The disclosure relates to a system and a method for enhancing user experience of an electronic device during abnormal sensation in a user body. The method includes determining, by a detection module, a level of abnormal sensation in the user body, generating, by a prevention module, vibrations of required frequency in a wearable device and in the electronic device, and performing, by an event modulation module, one or more functions for enhancing the user experience of the electronic device.

### CROSS-REFERENCE TO RELATED APPLICATION(S)

This application is a continuation application, claiming priority under § 365 (c), of an International application No. PCT/KR2023/003895, filed on Mar. 23, 2023, which is based on and claims the benefit of an Indian patent application number 202211034851, filed on Jun. 17, 2022, in the Indian Intellectual Property Office, the disclosure of which is incorporated by reference herein in its entirety.

### BACKGROUND

### 1. Field

The disclosure relates to detecting and preventing abnormal sensation in a user body. More particularly, the disclosure relates to a system and method for enhancing user experience of an electronic device during abnormal sensation in the user body.

### 2. Description of Related Art

Abnormal sensation is when a user feels tingling, nerve in-sensation, or numbness anywhere on his body. The abnormal sensation most commonly occurs in fingers, hands, arms, legs, or feet. It is normally painless and is caused by poor blood circulation in a user body. There can be various reasons behind the poor blood circulation in the user body like abnormal blood viscosity, external factors, or the like.

Most people experience the abnormal sensation due to external factors, such as bad posture while sitting, standing, or sleeping on an arm crooked under their head, or even due to wearing tight clothing for too long. These external factors generally create some pressure on nerves or blood vessels, which causes the abnormal sensation in the user body. Sometimes, during occurrence of the abnormal sensation, it becomes difficult for a user to work with the affected body part, which may hamper everyday activities. For example, hands affected by the abnormal sensation can make holding, typing, or operating of an electronic device hard or impossible.

The abnormal sensation is rarely disabling or permanent and go away upon reducing or relieving the pressure on the nerves. However, frequent occurrence of the abnormal sensation can expose the increased risk of developing long-term health hazards.

At present, there are several methods and systems for monitoring the abnormal sensation in the user body. However, the existing systems and methods are not able to enhance the user experience of the electronic device by modulating properties of the electronic device during the abnormal sensation. Further, the existing methods and devices are silent about utilizing a multi-device usage, such as a wearable device and the electronic device for preventing the abnormal sensation in the user body and generating recommendations to the user accordingly to prevent the abnormal sensation in future.

Therefore, in light of the foregoing discussion, there exists a need to overcome the aforementioned drawbacks caused by the abnormal sensation in the user body.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a method and a system for enhancing user experience of an electronic device during abnormal sensation in a user body are provided.

Additional aspects will be set forth in part in the description which follows and, in part, will be apparent from the description, or may be learned by practice of the presented embodiments.

In accordance with an aspect of the disclosure, a method for enhancing user experience of an electronic device during abnormal sensation in a user body is provided. The method includes determining, by a detection module, a level of abnormal sensation in the user body, generating, by a prevention module, vibrations of required frequency in a wearable device and in the electronic device, and performing, by an event modulation module, one or more functions for enhancing the user experience of the electronic device.

The abnormal sensation includes nerve in-sensation, tingling, or numbness. In an embodiment of the disclosure, the level of abnormal sensation is determined using one or more posture types of the user body, changes in one or more bio-markers and external parameters including at least but not limited to time duration of the one or more posture types.

In one embodiment, the electronic device includes at least but not limited to a mobile phone, personal digital assistant (PDA), computer, laptop, notebook, and camera and the wearable device includes at least but not limited to the electronic device that is worn as an accessory, embedded in clothing, implanted in the user's body, or tattooed on the skin, wristband, and wristwatch. The method further includes performing one or more functions for enhancing the user experience of the electronic device. In an embodiment of the disclosure, the one or more functions include identifying current window of the electronic device, extracting list of one or more features of the electronic device in the identified current window, identifying one or more features of the electronic device that degrades the user experience during the abnormal sensation, determining available alternate one or more features of the electronic device, and modulating the one or more features of the electronic device by disabling the identified one or more features of the electronic device that degrades the user experience and enabling the available alternate one or more features.

In accordance with another aspect of the disclosure, a system for enhancing user experience of an electronic device during abnormal sensation in a user body is provided. The system includes a detection module configured to determine a level of abnormal sensation in the user body, a prevention module, in communication with the detection module, configured to generate vibrations of required frequency in a wearable device and in the electronic device, and an event modulation module in communication with the prevention module configured to enhance the user experience of the electronic device.

Advantageously, the system further includes a recommendation module configured to provide one or more recommendations to a user, using an artificial intelligence based on frequency of occurrence of the abnormal sensation, one or more posture types of the user body, duration of the abnormal sensation and past abnormal sensation history of the user body, wherein the one or more recommendations include measures to prevent the abnormal sensation in future and/or recommendations based on the one or more functions performed by the event modulation module.

In an embodiment of the disclosure, the detection module includes a movement classification sub-module, biomarkers identification sub-module, and an abnormal sensation detection sub-module. The movement classification sub-module is configured to classify one or more movements of the user body in any one of one or more posture types of the user body which include type A, type B, type C, and type D. The biomarkers identification sub-module is configured to identify changes in one or more bio-markers with respect to time. The one or more bio-markers include blood volume, blood flow rate, and heart rate, which are calculated from one or more key points identified on noiseless optical signal. The abnormal sensation detection sub-module is configured to determine the level of abnormal sensation in the user body using the classified one or more posture types, identified change in one or more bio-markers and external parameters associated with the abnormal sensation. In an embodiment of the disclosure, the external parameters include at least but not limited to time duration of the one or more posture types.

In an embodiment of the disclosure, the one or more posture types of the user body specifically of hand include type A for pressed hand posture, type B for anti-gravity posture, type C for user induced numbness, and type D for vibrating hand syndrome.

In an embodiment of the disclosure, the one or more key points on the noiseless optical signals is identified by performing local maxima scalogram (LMS) matrix of the noiseless optical signal and successively row wise summation of each and every LMS matrix, wherein the noiseless optical signal is generated by filtering a body reflected optical signal received from the one or more sensors. Successively, all elements from original LMS matrix are removed to perform LMS rescaling. Thereafter, a peak detection analysis of the noiseless optical signal is performed for identifying the one or more key points including starting point and the systolic peak, wherein the peak detection analysis includes performing column-wise standard deviation for finding one or more indices having standard deviation equals to one.

In an embodiment of the disclosure, the prevention module includes a vibration intensity determination sub-module configured to determine vibration of required frequency and generating vibrations of required frequency in the wearable device and the electronic device. The prevention module further includes a vibration position identification sub-module configured to generate vibrations of required frequency in a localized region on display of the electronic device, wherein the localized region is identified by computing length of swipe arc, made by touch of finger of the user body on the display of the electronic device, using length of the finger computed from positional co-ordinates of first touch point and last touch point on the display.

In an embodiment of the disclosure, the event modulation module is configured to perform the one or more functions which include identifying current window of the electronic device, extract list of one or more features of the electronic device in the identified current window, identify one or more features of the electronic device that degrades the user experience during the abnormal sensation, determine available alternate one or more features of the electronic device, and modulate the one or more features of the electronic device by disabling the identified one or more features of the electronic device that degrade the user experience and enabling the available alternate one or more features.

In accordance with another aspect of the disclosure, one or more non-transitory computer-readable storage media storing computer-executable instructions that, when executed by a processor individually or collectively, cause an electronic device to perform operations are provided. The operations include enhancing user experience of the electronic device during abnormal sensation in a user body, determining, by a detection module, a level of abnormal sensation in the user body, generating, by a prevention module, vibrations of required frequency in a wearable device and in the electronic device, and performing, by an event modulation module, one or more functions for enhancing the user experience of the electronic device.

In an embodiment of the disclosure, the list of the one or more features of the electronic device includes at least but not limited to fingerprint authentication, face authentication, voice commands, iris authentication, text auto correction, haptic feedback for predictive text, customized keyboard, and auto size variation of keyboard.

Other aspects, advantages, and salient features will become apparent to those skilled in the art from the following detailed description, which, taken in conjunction with the annexed drawings, discloses various embodiments of the disclosure.

### DESCRIPTION OF THE DRAWINGS

The above and other aspects, features, and advantages of certain embodiments of the disclosure will be more apparent from the following description taken in conjunction with the accompanying drawings, in which:

FIG. 1 depicts a flow diagram illustrating a method for enhancing user experience of an electronic device during abnormal sensation in a user body according to an embodiment of the disclosure;

FIG. 2 depicts a block diagram of a system for enhancing a user experience of an electronic device during an abnormal sensation in a user body according to an embodiment of the disclosure;

FIG. 3 depicts a flow diagram illustrating a method of operation of a detection module for determining level of abnormal sensation in a user body according to an embodiment of the disclosure;

FIG. 4A depicts a flow diagram illustrating a method of operation of a movement classification sub-module for performing classification of one or more movements of a user body according to an embodiment of the disclosure;

FIG. 4B depicts a flow diagram illustrating a rule-based classifier for an extracted features according to an embodiment of the disclosure;

FIG. 4C depicts a pictorial representation of one or more posture types of hand according to an embodiment of the disclosure;

FIG. 5 depicts a flow diagram illustrating a method for identifying one or more key points on noiseless optical signals according to an embodiment of the disclosure;

FIG. 6A depicts a flow diagram illustrating a method of operation of prevention module for generating vibrations of required frequency in a wearable device and an electronic device according to an embodiment of the disclosure;

FIG. 6B depicts a pictorial representation of an electronic device showing positional co-ordinates of swipe arc for computing localized region on display, during abnormal sensation, according to an embodiment of the disclosure;

FIG. 7A depicts a flow diagram illustrating a method of operation of an event modulation module for modulating one or more features of an electronic device according to an embodiment of the disclosure;

FIG. 7B depicts a pictorial representation of classification performed using a support vector machine (SVM) learning model in an event modulation module for identification of one or more features of an electronic device that degrades a user experience of the electronic device according to an embodiment of the disclosure; and

FIG. 8 depicts a block diagram illustrating interconnection of a recommendation module with one or more modules of a system in order to provide recommendations to a user according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Furthermore, in the description, references to “an embodiment” or “an embodiment” means that a particular feature, structure, or characteristic described in connection with the embodiment is included in at least an embodiment of the disclosure. The appearance of the phrase “in an embodiment” in various places in the specification is not necessarily referring to the same embodiment of the disclosure, nor are separate or alternative embodiments mutually exclusive of other embodiments. Further, the terms “a” and “an” used herein do not denote a limitation of quantity, but rather denote the presence of at least one of the referenced items. Moreover, various features are described which may be exhibited by some embodiments and not by others. Similarly, various requirements are described, which may be requirements for some embodiments but not for other embodiments.

It should be appreciated that the blocks in each flowchart and combinations of the flowcharts may be performed by one or more computer programs which include computer-executable instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g., a central processing unit (CPU)), a communication processor (CP, e.g., a modem), a graphical processing unit (GPU), a neural processing unit (NPU) (e.g., an artificial intelligence (AI) chip), a wireless-fidelity (Wi-Fi) chip, a Bluetooth™ chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

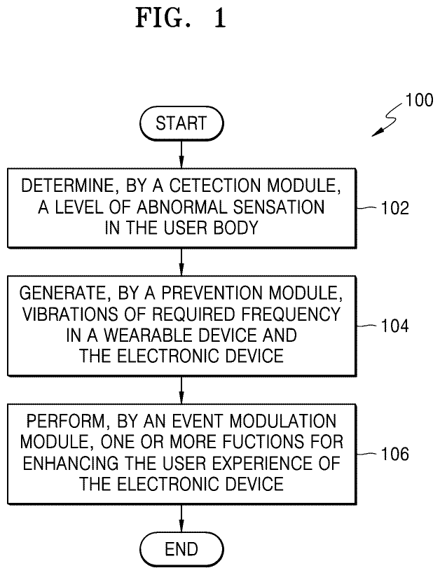

FIG. 1 depicts a flow diagram illustrating a method for enhancing a user experience of an electronic device during abnormal sensation in a user body according to an embodiment of the disclosure.

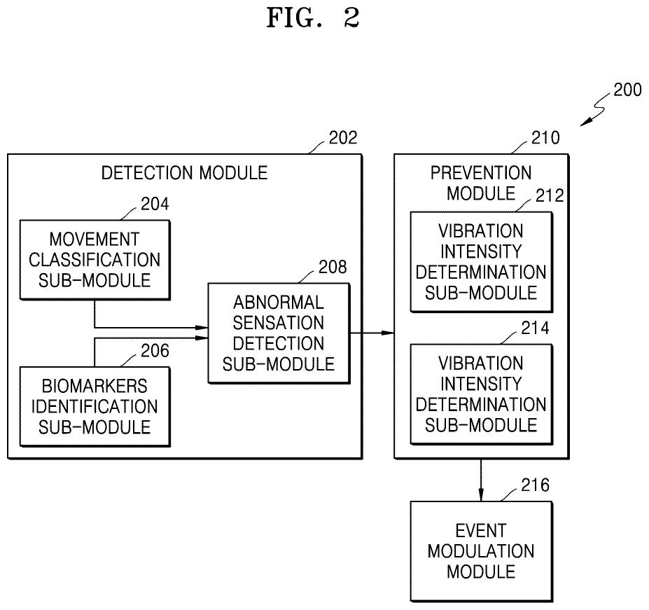

Referring to FIG. 1, a flow diagram showing a method 100 for enhancing user experience of an electronic device during abnormal sensation in a user body is disclosed. The method may be explained in conjunction with the system disclosed in FIG. 2. In the flow diagram, each block may represent a module, segment, or portion of code, which comprises one or more executable instructions for implementing the specified logical function(s). It should also be noted that in some alternative implementations, the functions noted in the blocks may occur out of the order noted in the drawings. For example, two blocks shown in succession in FIG. 1 may be executed substantially concurrently or the blocks may sometimes be executed in the reverse order, depending upon the functionality involved. Any process descriptions or blocks in flowcharts should be understood as representing modules, segments, or portions of code that include one or more executable instructions for implementing specific logical functions or operations in the process, and alternate implementations are included within the scope of the example embodiments in which functions may be executed out of order from that shown or discussed, including substantially concurrently or in reverse order, depending on the functionality involved. In addition, the process descriptions or blocks in flow charts should be understood as representing decisions made by a hardware structure, such as a state machine. The flow diagram starts at operation 102 and proceeds to operation 106.

At operation 102, a level of abnormal sensation is determined in the user body. In an embodiment of the disclosure, the level of abnormal sensation is determined by a detection module 202. The level of abnormal sensation in the user body is determined using classified one or more posture types, identified changes in one or more bio-markers and external parameters associated with the abnormal sensation including at least but not limited to time duration of the one or more posture types.

Successively, vibrations of required frequency is generated, at operation 104, in a wearable device and in the electronic device. In various embodiments of the disclosure, the electronic device includes at least but not limited to a mobile phone, PDA, computer, laptop, notebook, and camera and the wearable device includes at least but not limited to the electronic device that is worn as an accessory, embedded in clothing, implanted in the user's body, or tattooed on the skin, wristband, and wristwatch. The vibrations of required frequency is generated by a prevention module 210. In an embodiment of the disclosure, the prevention module 210 determines vibration of required frequency in the user body using a vibration intensity determination sub-module 212 and generates vibration of required frequency in the wearable device and in the electronic device. Further, the prevention module 210 is configured to generate vibrations of required frequency in a localized region on display of the electronic device, using a vibration position identification sub-module 214. In an embodiment of the disclosure, the localized region is identified by computing length of swipe arc, made by touch of finger of the user body on the display of the electronic device, using length of the finger computed from positional co-ordinates of first touch point and last touch point on the display.

Thereafter, one or more functions are performed, at operation 106, for enhancing the user experience of the electronic device. In an embodiment of the disclosure, the one or more functions are performed by an event modulation module 216. The one or more functions includes identifying current window of the electronic device, extracting list of one or more features of the electronic device in the identified current window, identifying one or more features of the electronic device that degrades the user experience, determining available alternate one or more features and modulating the one or more features of the electronic device for enhancing the user experience of the electronic device.

FIG. 2 depicts a block diagram of a system for enhancing a user experience of an electronic device during an abnormal sensation in a user body according to an embodiment of the disclosure.

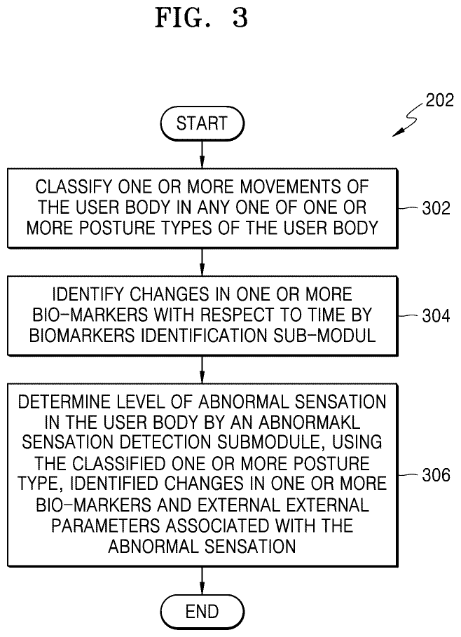

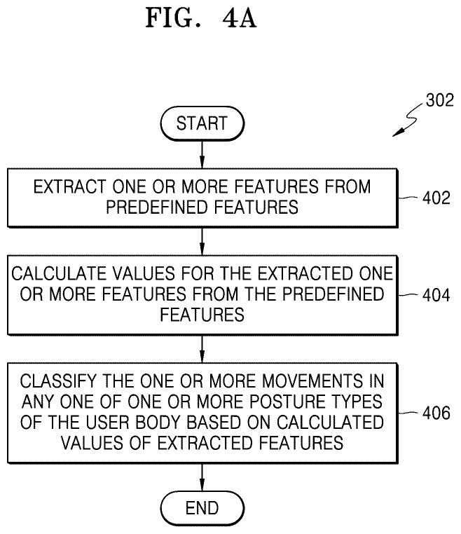

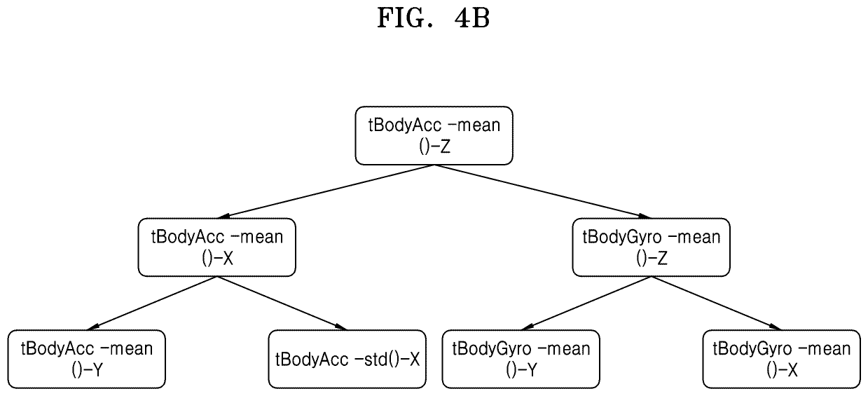

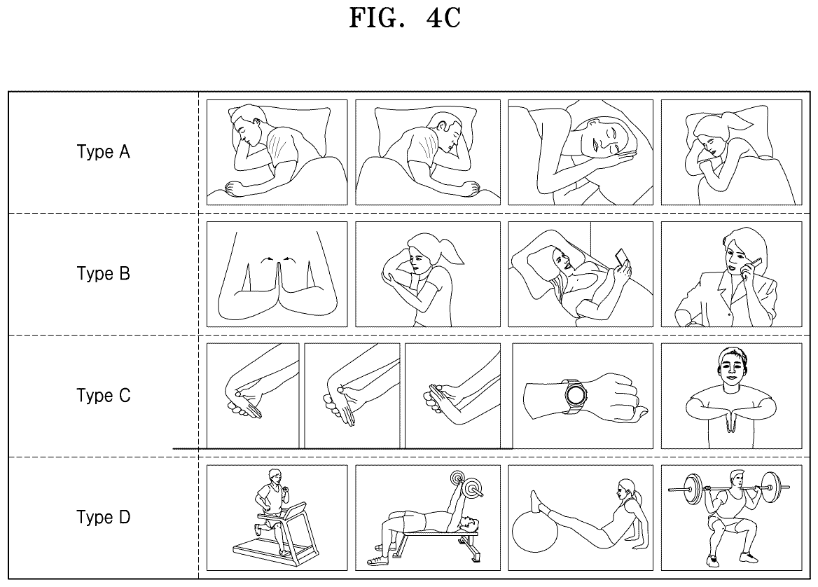

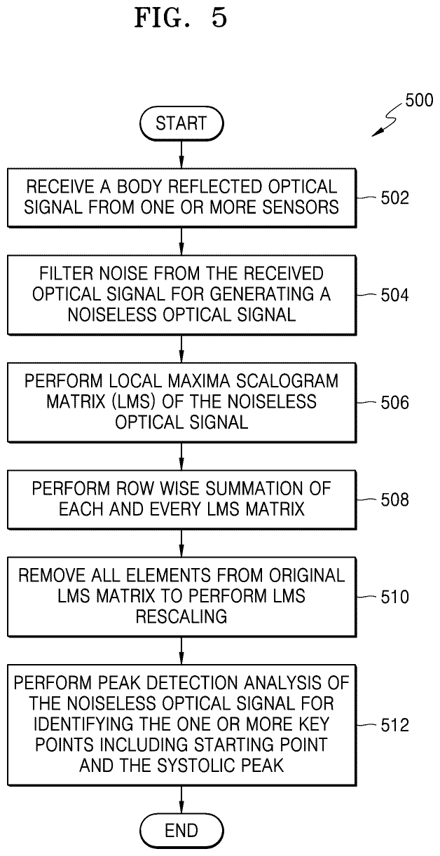

Referring to FIG. 2, a block diagram of a system 200 for enhancing the user experience of the electronic device during abnormal sensation in the user body is disclosed, in accordance with various embodiments of the disclosure. The system 200 comprises a detection module 202 for determining the level of abnormal sensation in the user body, which is described in FIG. 3. In an embodiment of the disclosure, the detection module 202 includes a movement 20 classification sub-module 204, a biomarkers identification sub-module 206, and an abnormal sensation detection sub-module 208. The movement classification sub-module 204 is configured for classifying the one or more movements of the user body in any one of one or more posture types of the user body, which is described in FIGS. 4A, 4B, and 4C. The biomarkers identification sub-module 206 is configured for identifying changes in one or more bio-markers with respect to time. The one or more bio-markers include at least but not limited to blood volume, blood flow rate, and heart rate, which are calculated from one or more key points identified on noiseless optical signal. The method for identifying the one or more key points on the noiseless optical signals is described in FIG. 5. The detection module 202 further includes an abnormal sensation detection sub-module 208 for determining the level of abnormal sensation in the user body. The abnormal sensation detection sub-module 208 utilizes the classified one or more posture types from the movement classification sub-module 204, identified changes in one or more bio-markers from the biomarkers identification sub-module 206 and external parameters associated with the abnormal sensation including at least but not limited to time duration of the one or more posture types in order to determine the level of abnormal sensation in the user body. In an embodiment of the disclosure, the abnormal sensation detection sub-module 208 utilizes a Tensorlfow's Keras sequential model, which uses an Adam optimizer with Sparse Categorical Cross entropy loss and accuracy metrics for determining the level of abnormal sensation in the user body. (https://www.tensorflow.org/)

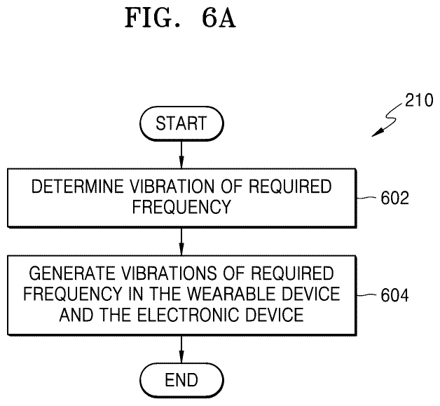

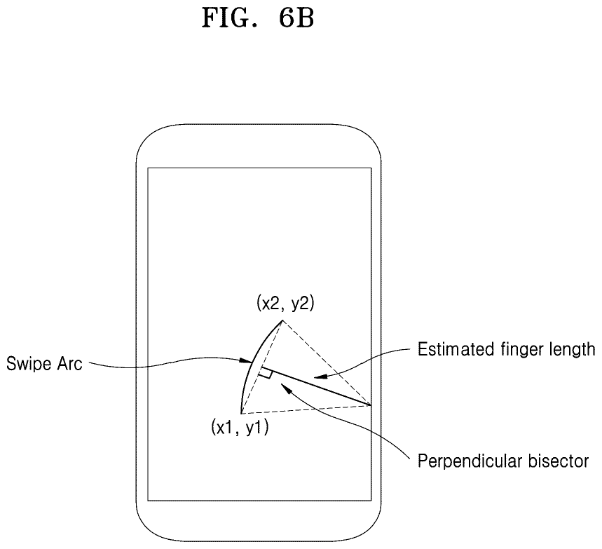

The system 200 further comprises a prevention module 210 for generating vibrations of required frequency in the wearable device and the electronic device. The prevention module 210 includes a vibration intensity determination sub-module 212 and a vibration position identification sub-module 214. In an embodiment of the disclosure, the vibration intensity determination sub-module 212 is configured to determine vibration of required frequency using the level of abnormal sensation in the user body received from the detection module 202 and one or more health parameters which include age, ambient temperature, and diabetic status of the user body and generating vibrations of required frequency in the wearable device and the electronic device. In an embodiment of the disclosure, the one or more health parameters may be collected from the wearable device or a computing device. The detailed method of operation of prevention module for generating vibrations of required frequency in the wearable device and the electronic device is explained in FIG. 6A. The vibration position identification sub module 214 is configured for generating vibrations of required frequency in a localized region on display of the electronic device. In an embodiment of the disclosure, the localized region is identified by computing length of swipe arc, made by touch of finger of the user body on the display of the electronic device. In an embodiment of the disclosure, the length of the swipe arc is computed using length of the finger computed from positional co-ordinates of first touch point and last touch point on the display, which is described in FIG. 6B.

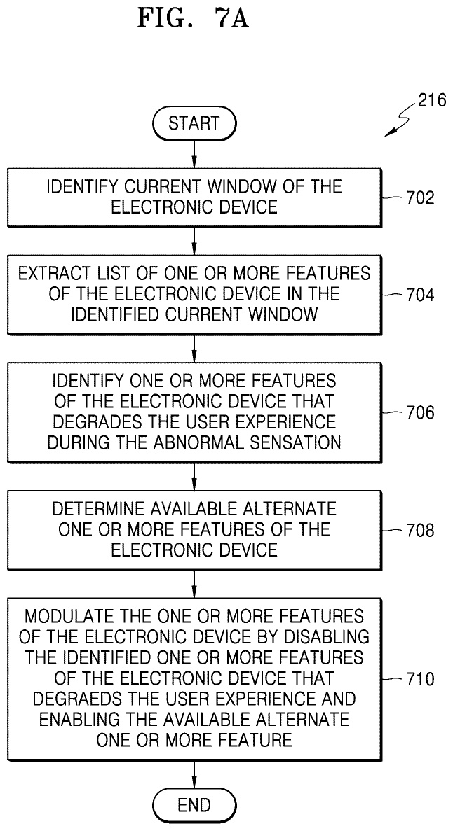

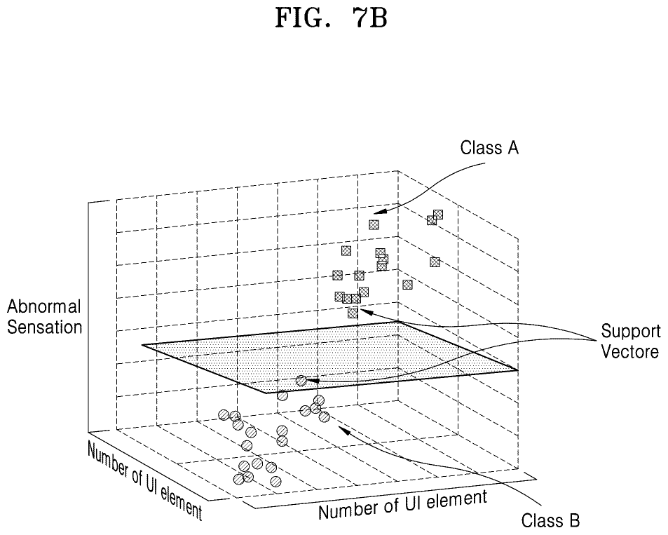

The system 200 further comprises an event modulation module 216 for performing one or more functions for enhancing the user experience of the electronic device. The one or more functions include identifying current window of the electronic device, extracting list of one or more features of the electronic device in the identified current window, identifying one or more features of the electronic device that degrade the user experience, determining available alternate one or more features and modulating the one or more features of the electronic device for enhancing the user experience of the electronic device, which is described in FIGS. 7A and 7B.

FIG. 3 depicts a flow diagram illustrating a method of operation of a detection module for determining level of abnormal sensation in the user body according to an embodiment of the disclosure.

Referring to FIG. 3, a flow diagram showing a method of operation of the detection module 202 for determining the level of abnormal sensation in the user body is disclosed. In an illustrated embodiment of the disclosure, one or more movements of the user body is classified in any one of one or more posture types of the user body, at operation 302, by a movement classification sub-module 204. The classification of the one or more movements of the user body in any one of one or more posture types of the user body is explained in conjunction with FIGS. 4A, 4B, and 4C.

FIG. 4A depicts a flow diagram illustrating a method of operation of a movement classification sub-module for performing classification of one or more movements of a user body according to an embodiment of the disclosure.

Referring to FIG. 4A, the movement classification sub-module 204 extracts one or more features from predefined features, at operation 402. The predefined features are those features of the user body which are defined for devices, such as gyroscope and accelerometer for measuring one or more movements of the user body. In an embodiment of the disclosure, twelve features are extracted from six pre-defined features by the movement classification sub-module 204 as shown below in Table 1.

TABLE 1

| S. | Pre-defined | Extracted One Or More |
|---|---|---|
| No. | Features | Features |
| 1 | tBodyAcc-X | tBody Acc-mean()-X |
| 2 | tBodyAcc-Y | tBody Acc-mean()-Y |
| 3 | tBodyAcc-Z | tBody Acc-mean()-Z |
| 4 | tBodyGyro-X | tBodyGyro-mean()-X |
| 5 | tBodyGyro-Y | tBodyGyro-mean()-Y |
| 6 | tBodyGyro-Z | tBodyGyro-mean()-Z |
| 7 | | tBodyAcc-std()-X |
| 8 | | tBody Acc-std()-Y |
| 9 | | tBodyAcc-std()-Z |
| 10 | | tBodyGyro-std()-X |
| 11 | | tBodyGyro-std()-Y |
| 12 | | tBodyGyro-std()-Z |

After successful extraction, values for the extracted one or more features are calculated, at operation 404, by the movement classification sub-module 204. The values for the extracted one or more features are calculated from the pre-defined features using the Equation 1 and Equation 2 given below:

$$Mean = \sum \frac{{x}_{i}}{n} \qquad Equation 1$$

$${S}_{X} = \sqrt{\frac{{\sum }_{1 = 1}^{n} {( {x}_{i} - \overline{x} )}^{2}}{n - 1}} \qquad Equation 2$$

Wherein n is the number of data points, xᵢ is each of the values of the data, and x with ‘-’ is the mean of xᵢ.

After calculating values for the one or more features, classification of the one or more movements in any one of one or more posture types of the user body is performed at operation 406, based on the values of the one or more extracted features for the respective one or more movements of the user body.

In an embodiment of the disclosure, the movement classification sub-module 204 utilizes a classifier model to perform classification of the one or more movements of the user body. In an embodiment of the disclosure, a total of fifty classifiers with maximum depth of six are used to train the classifier model. The outputs of these fifty classifiers are used to determine the classification of the one or more movements. One of the rule based classifier for the extracted features is shown in FIG. 4B.

FIG. 4B depicts a flow diagram illustrating a rule-based classifier for extracted features according to an embodiment of the disclosure.

Referring to FIG. 4B, the classifier model computes movement classification for dataset disclosed in Table 2 which is shown below. Table 2 shows sample dataset for movement classification.

TABLE 2

| tBodyAcc- | tBodyAcc- | tBodyAcc- | tBodyAcc- | tBodyAcc- | tBodyAcc- | | Posture |
|---|---|---|---|---|---|---|---|
| mean-X | mean-Y | mean-Z | std-X | std-Y | std-Z | Class | Type |
| 0.275 | −0.010 | −0.099 | −0.998 | −0.986 | −0.991 | Numbness | Type A |
| 0.278 | −0.015 | −0.098 | −0.998 | −0.981 | −0.991 | Non- | NA |
| | | | | | | Numbness | |
| 0.279 | −0.021 | −0.109 | −0.997 | −0.992 | −0.985 | Numbness | Type A |
| 0.274 | −0.023 | −0.112 | −0.996 | −0.991 | −0.987 | Numbness | Type C |
| 0.269 | −0.027 | −0.110 | −0.996 | −0.986 | −0.988 | Numbness | Type A |
| 0.275 | −0.018 | −0.097 | −0.996 | −0.968 | −0.980 | Non- | NA |
| | | | | | | Numbness | |
| 0.281 | −0.004 | −0.086 | −0.989 | −0.959 | −0.973 | Numbness | Type B |
| 0.297 | −0.023 | 0.021 | −0.952 | −0.630 | −0.323 | Non- | NA |
| | | | | | | Numbness | |
| 0.265 | 0.010 | −0.170 | −0.988 | −0.874 | −0.838 | Numbness | Type B |
| 0.279 | −0.023 | −0.092 | −0.994 | −0.958 | −0.957 | Non- | NA |
| | | | | | | Numbness | |
| 0.162 | −0.122 | 0.137 | −0.872 | −0.523 | −0.356 | Non- | NA |
| | | | | | | Numbness | |
| 0.221 | −0.087 | 0.044 | −0.810 | −0.305 | −.0032 | Non- | NA |
| | | | | | | Numbness | |
| 0.044 | −0.100 | 0.122 | −0.659 | −0.151 | 0.242 | Non- | NA |
| | | | | | | Numbness | |
| −0.325 | −0.196 | 0.494 | −0.723 | −0.296 | −0.096 | Numbness | Type C |
| −0.072 | −0.079 | 0.183 | −0.766 | −0.588 | −0.341 | Numbness | Type C |

The classifier model classifies movement classification in any one of the one or more posture types by calculating impurity for a sub-dataset of the dataset disclosed in Table 2, using the Equation 3 given below. Equation 3 shows how to calculate impurity of a sub-dataset.

$$Imp = {a}^{2} ⋆ {b}^{2} \qquad Equation 3$$

Wherein a is the number of ‘zero's and b is the number of ‘1's.

After calculating the impurity for every column and every possible value in respective column, the impurity with maximum score is chosen as a splitting rule at a node. Iteratively, same process is repeated until height of tree>=Max_depth (user input) OR perfect split is achieved with score 1.0.

As shown in Table 2, the one or more movements of the user body is classified in any one of the one or more posture types. The one or more posture types of the user body include type A, type B, type C, and type D. In an embodiment of the disclosure, the type A, type B, type C, and type D of the one or more posture types of the user body specifically of hand represents pressed hand posture, anti-gravity posture, user induced numbness, and vibrating hand syndrome respectively as shown in FIG. 4C.

FIG. 4C depicts a pictorial representation of one or more posture types of hand according to an embodiment of the disclosure.

Referring to FIG. 4C, type A is related to hand pressed posture. In FIG. 4C, type B is related to anti-gravity posture. In FIG. 4C, type C is related to user-induced numbness. For example, wrong hand placement or tightly coupled smart watch could induce numbness. In FIG. 4C, type D is related to vibrating hand syndrome.

Successively, changes in one or more bio-markers are identified with respect to time, at operation 304, by biomarkers identification sub-module 206. The one or more bio-markers include at least but not limited to the blood volume, blood flow rate, and heart rate, which are calculated from one or more key points identified on noiseless optical signal. The identification of one or more key points on noiseless optical signal is explained in conjunction with FIG. 5.

FIG. 5 depicts a flow diagram 500 illustrating a method for identifying one or more key points on noiseless optical signals according to an embodiment of the disclosure.

Referring to FIG. 5, a body reflected optical signal is received, at operation 502, from the one or more sensors. The one or more sensors includes an optical sensor, a PPG sensor, and a camera sensor. After receiving the body reflected optical signal, filtration is performed, at operation 504, for generating a noiseless optical signal. Further, local maxima scalogram (LMS) matrix of the noiseless optical signal is performed, at operation 506. In an embodiment of the disclosure, the LMS matrix of the noiseless optical signal is performed as Equation 4 and Equation 5.

$$M = ( \begin{matrix}{m}_{1 , 1} & {m}_{1 , 2} & … & {m}_{1 , N} \\ {m}_{2 , 1} & {m}_{2 , 2} & … & {m}_{2 , N} \\ ⋮ & ⋮ & ⋱ & ⋮ \\ {m}_{L , 1} & {m}_{L , 2} & … & {m}_{L , N}\end{matrix} ) = ( {m}_{k , i} ) \qquad Equation 4$$

$${m}_{k , i} = \{ \begin{matrix}0 , & {x}_{i - 1} > {x}_{i - k - 1} ∧ {x}_{i - 1} > {x}_{i - k - 1} \\ r + α , & otherwise\end{matrix} \qquad Equation 5$$

Wherein r is a uniformly distributed random number in the range of [0, 1], α is a constant factor which is 1, and moving window is determined using Equation 6.

$$\begin{matrix}\{ {w}_{k} = 2 k \vert k = 1 , 2 , … , L \} & L = ⌈ N / 2 ⌉\end{matrix} - 1 \qquad Equation 6$$

Further, row wise summation of each and every LMS matrix is performed at operation 508 using Equation 7.

$${γ}_{k} = {\sum }_{i = 1}^{N} {m}_{k , \text{?}} , for k ϵ \{ 1 , 2 , … , L \} \qquad Equation 7$$

$$\text{?} \text{indicates text missing or illegible when filed}$$

In an embodiment of the disclosure, the row wise summation depends upon window size, which is defined as Equation 8.

$$γ = [ {γ}_{1} , {γ}_{2} , … , {γ}_{i} , … , {γ}_{L} ] \qquad Equation 8$$

Further, final window size is window having a maximum number of local maxima. Therefore, global maxima is defined as Equation 9.

$$λ = Max ( {γ}_{k} ) \qquad Equation 9$$

Further, LMS rescaling is performed, at operation 510, by removing all elements from the original LMS matrix. In an embodiment of the disclosure, all the elements for which k<λ are removed from original LMS matrix.

Then, peak detection analysis of the noiseless optical signal is performed at operation 512, for identifying the one or more key points including starting point and the systolic peak. The peak detection analysis includes performing column-wise standard deviation for finding one or more indices having standard deviation using Equation 10.

$${σ}_{\text{?}} = \frac{1}{λ - 1} {\sum }_{\text{?}}^{\text{?}} {[ {( {m}_{\text{?}} - \frac{1}{λ} {\sum }_{\text{?}}^{\text{?}} {m}_{\text{?}} )}^{2} ]}^{\frac{1}{2}} , for i ϵ \{ 1 , 2 , … , N \} \qquad Equation 10$$

$$\text{?} \text{indicates text missing or illegible when filed}$$

After successful identification of the one or more key points, one or more bio-markers are computed. Table 3 shows a sample dataset for one or more bio-markers specifically heart rate and blood flow rate. Table 3 shows sample dataset for changes in one or more bio-markers.

TABLE 3

| Biomarkers at T1 | Biomarkers at T2 | Effective change |
|---|---|---|
| HR = 90QR = 8 | HR = 70 | HR = −22% |
| | QR = 6 | QR = −25% |
| HR = 70QR = 7.9 | HR = 52 | HR = −24% |
| | QR = 6.1 | QR = −22% |
| HR = 84QR = 5.8 | HR = 80 | HR = −4% |
| | QR = 6.1 | QR = +5% |
| HR = 81QR = 6.2 | HR = 70 | HR = −15% |
| | QR = 4.1 | QR = −30% |

As shown in Table 3, Photoplethysmography (PPG) signal are received at two different time period T1 and T2. Further, values for two bio-markers i.e., blood flow rate (referred in the table 2 as Q) and heart rate (referred in the table 2 as HR) are computed for the two different time period T1 and T2 in order to identify changes in blood flow rate (Q) and heart rate (HR).

The blood flow rate is referred as movement of blood through vessels, which represents blood circulation in any local region of the body. In an embodiment of the disclosure, the blood flow rate is obtained from the one or more key points identified on the received PPG signals. Blood flow rate (Q) is blood volume/Crest time. Blood volume is an area under the curve up to Systole. Systole represents period of contraction of the ventricles, it means ejection of blood from heart i.e., area up to systole peak. If area is more than that means more blood is flowing. In an embodiment of the disclosure, the heart rate is obtained from the one or more key points identified on the received PPG signals. In another embodiment of the disclosure, the heart rate is obtained from the wearable device.

Thereafter, the level of abnormal sensation in the user body is determined by an abnormal sensation detection sub-module 208, at operation 306, using the one or more posture types classified by the movement classification sub-module 204, changes in one or more bio-markers identified by the biomarkers identification sub-module 206, and external parameters associated with the abnormal sensation including at least but not limited to time duration of the one or more posture types. Table 4 shows the sample dataset for the level of abnormal sensation in the user body.

TABLE 4

| | Posture | | | Abnormal |
|---|---|---|---|---|
| Posture | Duration | Change | Change | Sensation |
| Type | (seconds) | in Q | in HR | level |
| A | 600 | −22& | −19% | 8 |
| C | 100 | −6% | +4% | 0 |
| B | 285 | −18% | +12% | 7 |
| A | 139 | −14% | −17% | 1 |
| D | 120 | −5% | −3% | 6 |
| C | 5829 | −6% | −2% | 9 |
| A | 486 | −19% | −14% | 5 |

As shown in the Table 4, the level of abnormal sensation depends upon the one or more posture types, time duration of the one or more posture types, change in blood flow rate (Q) and change in heart rate (HR). In an embodiment of the disclosure, the abnormal sensation detection sub-module 208 utilizes a Tensorlfow's Keras sequential model, which uses an Adam optimizer with SparseCategoricalCrossentropy loss and accuracy metrics for determining the level of abnormal sensation. In an embodiment of the disclosure, the model is used with input shape (15,1), output shape (K,1), where K is the number of coordinate blocks taken, and 3 is number of dense layers. The model outputs the abnormal sensation level on a scale of 1-10.

FIG. 6A depicts a flow diagram illustrating a method of operation of prevention module for generating vibrations of required frequency in a wearable device and an electronic device according to an embodiment of the disclosure.

Referring to FIG. 6A, a flow diagram showing a method of operation of prevention module for generating vibrations of required frequency in the wearable device and the electronic device is disclosed, in accordance with various embodiments of the disclosure. The prevention module includes two sub-modules one is vibration intensity determination sub-module 212 and other is vibration position identification sub-module 214. In FIG. 6A, vibration of required frequency is determined, at operation 602. In an embodiment of the disclosure, the vibration of required frequency is determined by a vibration intensity determination sub-module 212 using the level of abnormal sensation in the user body from the detection module 202 and one or more health parameters which include age, ambient temperature, and diabetic status of the user body collected by a wearable device or a computing device. The computing device may be, for example, but is not limited to, an electronic, magnetic, optical, electromagnetic, infrared, or semiconductor system, apparatus, device, or any suitable combination of the foregoing.

The vibration intensity determination sub-module 212 utilizes a regression model to determine the vibration of required frequency. Table 5 shows a sample dataset for the vibration intensity determination.

TABLE 5

| Level of | | | | |
|---|---|---|---|---|
| Abnormal | | Ambient | | Vibration |
| Sensation | Age | temperature | Diabetic | Intensity |
| 2 | 25 | 10 | 0 | 31 |
| 7 | 40 | 5 | 1 | 44 |
| 4 | 30 | 40 | 0 | 35 |
| 1 | 18 | 24 | 0 | 31 |
| 6 | 55 | 30 | 1 | 35 |
| 8 | 70 | 22 | 1 | 40 |
| 6 | 56 | 10 | 1 | 40 |
| 9 | 84 | 33 | 1 | 44 |
| 9 | 59 | 15 | 1 | 44 |

As shown in Table 5, the diabetic column represents status of diabetes collected by the wearable device, value 0 represents diabetes and 1 represents non-diabetes, the vibration intensity column represents vibration of required frequency varies between 30-50 Hz which implies the vibrations between 30 to 50 Hz don't cause narrowing of the tissues during the recovery period.

In an embodiment of the disclosure, the frequency of vibration is computed by the regression model using the Equation 11 given below.

$$y = b 0 + b 1 * x 1 + b 2 * x 2 + b 3 * x 3 + b 4 * x 4 + ε \qquad Equation 11$$

Wherein y is the vibration of required frequency, x1 is the level of abnormal sensation, x2 the age of the user, x3 is the ambient temperature, x4 is diabetic status of the user, and & is an error arterial elasticity b0, b1, b2, and b3 are biases for adjusting coefficients to reduce error, wherein b0, b1, b2, and b3 are calculated using the Equation 12 given below.

$$\begin{matrix}{b}_{0} = Y - {b}_{1} {X}_{1} - {b}_{2} {X}_{2} - {b}_{3} {X}_{3} \\ {b}_{1} = \frac{( Σ {X}_{1}^{2} ) ( Σ {X}_{1} Y ) - ( Σ {X}_{1} {X}_{2} ) ( Σ {X}_{2} Y ) - ( Σ {X}_{1} {X}_{3} ) ( Σ {X}_{3} Y )}{( Σ {X}_{1}^{2} ) ( Σ {X}_{2}^{2} ) ( Σ {X}_{3}^{2} ) - ( Σ {X}_{1} {X}_{2} {X}_{3} )} \\ {b}_{2} = \frac{( Σ {X}_{2}^{2} ) ( Σ {X}_{2} Y ) - ( Σ {X}_{2} {X}_{3} ) ( Σ {X}_{3} Y ) - ( Σ {X}_{2} {X}_{1} ) ( Σ {X}_{1} Y )}{( Σ {X}_{1}^{2} ) ( Σ {X}_{2}^{2} ) ( Σ {X}_{3}^{2} ) - ( Σ {X}_{1} {X}_{2} {X}_{3} )} \\ {b}_{3} = \frac{( Σ {X}_{3}^{2} ) ( Σ {X}_{3} Y ) - ( Σ {X}_{3} {X}_{1} ) ( Σ {X}_{1} Y ) - ( Σ {X}_{3} {X}_{2} ) ( Σ {X}_{2} Y )}{( Σ {X}_{1}^{2} ) ( Σ {X}_{2}^{2} ) ( Σ {X}_{3}^{2} ) - ( Σ {X}_{1} {X}_{2} {X}_{3} )}\end{matrix} \qquad Equation 12$$

Further, root mean square error (RMSE) of the determined vibration of required frequency is calculated using the Equation 13 given below.

$$R M S E = \sqrt{{\sum }_{i = 1}^{n} \frac{{( {\hat{y}}_{i} - {y}_{i} )}^{2}}{n}} \qquad Equation 13$$

It should be noted that the RMSE is required to be minimized to attain more accuracy.

Successively, vibrations of required frequency is generated in the wearable device and in the electronic device, at operation 604, during the abnormal sensation.

The vibration position identification sub-module 214 identifies a localized region on display of the electronic device, wherein the localized region is identified by computing length of swipe arc which is explained in conjunction with FIG. 6B.

FIG. 6B illustrates swipe arc made by touch of finger of a user body during an abnormal sensation on a display of an electronic device according to an embodiment of the disclosure.

Referring to FIG. 6B, for computing the length of swipe arc, the vibration position identification sub-module 214 computes length of the finger from positional co-ordinates of first touch point and last touch point on the display. (x1, y1) shows the first touch point and (x2, y2) shows the last touch point. Mid-point is ((x1+x2)/2, (y1+y2)/2). Slope is (y2−y1)/(x2−x1). Perpendicular slope is −1/slope. Perpendicular bisector equation is defined as Equation 14.

$$y - {y}_{mid} = m ( x - {x}_{mid} ) \qquad Equation 14$$

Estimated length of finger is length of perpendicular bisector till edge of the display.

In an embodiment where (x1, y1) is (730, 1755) and (x2, y2) is (896, 1373), mid-point is (813, 1565), perpendicular slope is 0.4346, and perpendicular bisector equation is defined as y−1565=0.4346(x−813). Top of perpendicular bisector could be calculated by putting x=1080. By putting x=1080, y=1681.03. Therefore, estimated length of finger is calculated as 292.039314.

After calculating the length of swipe arc, vibrations in the localized region is generated. In an embodiment of the disclosure, the vibrations of required frequency may be generated by the vibration position identification sub-module 214. In another embodiment of the disclosure, the vibrations of required frequency may be generated by the wearable device.

FIG. 7A depicts a flow diagram illustrating a method of operation of an event modulation module for modulating one or more features of an electronic device according to an embodiment of the disclosure.

Referring to FIG. 7A, a method of operation of the event modulation module for modulating the one or more features of the electronic device is disclosed, in accordance with various embodiments of the disclosure. At first, current window of the electronic device is identified, at operation 702, by event modulation module 216.

Successively, list of one or more features of the electronic device are extracted, at operation 704, in the identified current window. In an embodiment of the disclosure, the list of the one or more features of the electronic device includes at least but not limited to fingerprint authentication, face authentication, voice commands, iris authentication, text auto correction, haptic feedback for predictive text, customized keyboard, and auto size variation of keyboard.

Successively, one or more features of the electronic device that degrades the user experience during the abnormal sensation are identified, at operation 706. In an embodiment of the disclosure, the one or more features which degrades the user experience of the electronic device are identified by utilizing a support vector machine (SVM) learning model. The SVM learning model searches hyperplanes with largest margin. Hyperplanes are decision boundaries that help classify the data points which fall on either side of the hyperplane and signify different classes. The classification of the one or more features of the electronic device in order to identify the one or more features degrading the user experience is explained in conjunction with FIG. 7B.

FIG. 7B depicts a pictorial representation of classification performed using a support vector machine (SVM) learning model in an event modulation module for identification of one or more features of an electronic device that degrades a user experience of the electronic device according to an embodiment of the disclosure.

Referring to FIG. 7B, the one or more features are represented as data points and classified in two classes one class is class A and another class is class B. The class A includes the data points impacted due to the abnormal sensation and positioned on upper space of the hyperplane. The class B includes the data points that are non-impacted due to the abnormal sensation and positioned on lower space of the hyperplane. Further, there are support vectors in both of the classes. The support vectors are the data points that are closer to the hyperplane and influence the position and orientation of the hyperplane.

In the SVM model, hinge loss function is used to maximize the margin between the data points and the hyperplane as Equation 15.

$$c ( x , y , f ( x ) ) = \{ \begin{matrix}0 , & if y ⋆ f ( x ) \geq 1 \\ 1 - y ⋆ f ( x ) , & else\end{matrix} \qquad Equation 15$$

Further, a regularization parameter is added to balance the margin maximization and loss. In an embodiment of the disclosure, the regularization parameter is a cost function which is Equation 16.

$${min}_{w} λ {‖ w ‖}^{2} + {\sum }_{i = 1}^{n} {( 1 - {y}_{i} 〈 {x}_{i} , w 〉 )}_{+} \qquad Equation 16$$

Further, gradients are determined by performing partial derivative of the cost function with respect to weights as Equation 17.

$$\frac{δ}{δ {w}_{k}} λ {‖ w ‖}^{2} = 2 λ {w}_{k} \frac{δ}{δ {w}_{k}} {( 1 - {y}_{i} 〈 {x}_{i} , w 〉 )}_{+} = \{ \begin{matrix}0 , & if {y}_{i} 〈 {x}_{i} , w 〉 \geq 1 \\ - {y}_{i} {x}_{ik} , & else\end{matrix} \qquad Equation 17$$

In an embodiment of the disclosure, weights are updated using the determined gradients. In one case, when there is no misclassification, gradient update is defined as Equation 18.

$$w = w - α \cdot ( 2 λ w ) \qquad Equation 18$$

In another case, when there is misclassification, gradient update is defined as Equation 19.

$$w = w + α \cdot ( {y}_{i} \cdot {x}_{i} - 2 λ w ) \qquad Equation 19$$

Successively, available alternate one or more features of the electronic device are determined, at operation 708. In an embodiment of the disclosure, the one or more features of the electronic device or the data points which are non-impacted due to abnormal sensation are taken as alternate one or more features of the electronic device by the event modulation module 216.

Thereafter, the one or more features of the electronic device are modulated, at operation 710, by disabling the identified one or more features of the electronic device that degrades the user experience and enabling the available alternate one or more features. Table 6 shows a sample dataset for the system event modulation.

TABLE 6

| Abnormal Sensation | | System Events |
|---|---|---|
| Detected | Activity Screen | Modulated |
| Yes | Lockscreen with | Fingerprint disabled |
| | fingerprint enabled | and Face |
| | | authentication |
| | | enabled |
| Yes | Home screen where | No modulation is |
| | user can scroll | system property |
| Yes | User is typing | Predictive text |
| | | functionality enabled |
| Yes | User trying to capture | Enlarge actionable |
| | Image | buttons |

As shown in Table 6, the one or more features of the electronic device, such as fingerprint, face authentications, predictive text functionality, actionable buttons, or the like, are modulated on detection of the abnormal sensation on the user body.

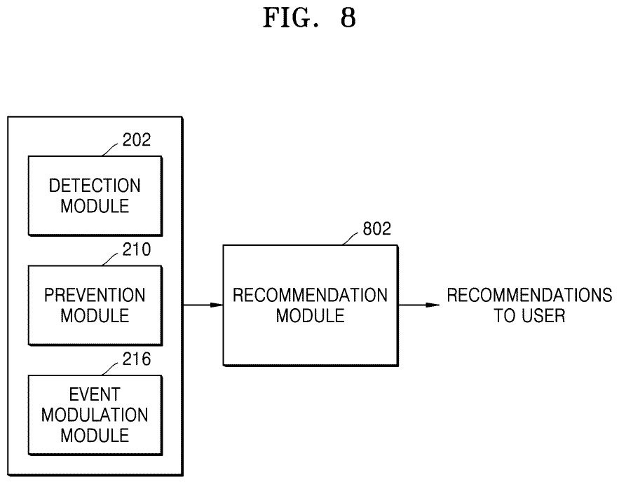

FIG. 8 depicts a block diagram illustrating interconnection of a recommendation module with one or more modules of a system in order to provide recommendations to a user according to an embodiment of the disclosure.

Referring to FIG. 8, a block diagram illustrating interconnection of a recommendation module with one or more modules of the system which includes the detection module, the prevention module, and the event modulation module in order to provide recommendations to the user is disclosed, in accordance with various embodiments of the disclosure. The recommendation module 802 is configured for receiving inputs from the one or more modules for providing measures the user needs to take to prevent the abnormal sensation in future. In another embodiment of the disclosure, the recommendation module 802 is configured for providing recommendation to the user based on the one or more functions performed by the event modulation module 216, to enhance the user experience of the electronic device. Table 7 shows a sample dataset for the recommendation module.

TABLE 7

| | | | Past numbness | |
|---|---|---|---|---|
| | Frequency | | history | |
| Posture | of | Numbness | {Numbness | |
| Type | occurrence | duration | level, duration} | Recommendation |
| A | 3 | 600 | {7, 300} | Doctor |
| | | | | consultation |
| | | | | recommended |
| C | 9 | 450 | {8,500} | Doctor |
| | | | | consultation |
| | | | | recommended |
| B | 2 | 80 | {3, 230} | Please |
| | | | | change hand |
| | | | | posture |
| A | 4 | 18 | {6, 50} | Please loosen |
| | | | | your watch |
| | | | | coupling |
| D | 7 | 90 | {4, 120} | Long time in |
| | | | | sam3e |
| | | | | position |
| C | 1 | 25 | {0, 0} | NA |
| A | 2 | 37 | {6, 50} | Change your |
| | | | | hand |
| | | | | alignment |

As shown in Table 7, the one or more recommendations include doctor consultation recommended, change hand posture, loose watch coupling, long time same position, or the like, based on inputs including one or more posture types of the user body, the frequency of occurrence of the abnormal sensation, duration of the abnormal sensation, and past abnormal sensation history of the user body received from the one or more modules. In an embodiment of the disclosure, the recommendation module 802 utilizes an artificial intelligence. In an embodiment of the disclosure, the reinforcement learning model is used for generating the one or more recommendations. The reinforcement learning model is configured to work by interacting with environment and provide recommendation using Equation 20 given below.

$$Q ( state , action ) = R ( state , action ) + γ ⋆ max [ Q ( next state , action ) ] \qquad Equation 20$$

Wherein state represents level of abnormal sensation, action represents recommendation generation, Q represents a matrix created for current state and action, which is memory of agent and stores learning of the agent through experience, R represents a reward function which takes a state and action and outputs a reward value, γ* represents a discount factor, which is defined as if discount factor is close to 0, then agent do not explore all actions and consider immediate awards, else explore all actions, and Q (next state, action) represents Q matrix for next state and action.

Further, the reward matrix is positive if the level of abnormal sensation is less than threshold value (β) as Table 8 shown below.

TABLE 8

| Reward Matrix | Equal or less than β | More than β |
|---|---|---|
| Level of Abnormal | +1 | −1 |
| Sensation | | |

In an embodiment of the disclosure, the threshold value may be a user defined value.

In an embodiment of the disclosure, when the user is wearing a tightly coupled watch for a longer duration and suddenly started feeling pain and abnormal sensation in the hand, then the recommendation module 802 may provide the recommendation to the user to couple the watch loosely by one point to prevent the pain and abnormal sensation in the hand. In an embodiment of the disclosure, when the user is trying to unlock the mobile phone with fingerprint, but not able to unlock due to abnormal sensation in the hand, then the recommendation module 802 may provide recommendation that the fingerprint is disabled and voice command is activated.

It has thus been seen that the system and method for enhancing user experience of an electronic device during abnormal sensation in a user body according to the disclosure achieve the purposes highlighted earlier. Such a system and method can in any case undergo numerous modifications and variants, all of which are covered by the same innovative concept, moreover, all of the details can be replaced by technically equivalent elements.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device, cause the electronic device to perform a method of the disclosure.

Any such software may be stored in the form of volatile or non-volatile storage, such as, for example, a storage device like read only memory (ROM), whether erasable or rewritable or not, or in the form of memory, such as, for example, random access memory (RAM), memory chips, device or integrated circuits or on an optically or magnetically readable medium, such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A method for enhancing user experience of an electronic device during abnormal sensation in a user body, the method comprising:
determining, by a detection module, a level of abnormal sensation in the user body;
generating, by a prevention module, vibrations of required frequency in a wearable device and in the electronic device; and
performing, by an event modulation module, one or more functions for enhancing the user experience of the electronic device.

2. The method of claim 1,
wherein the method comprises:
providing one or more recommendations to a user, by a recommendation module, using an artificial intelligence based on frequency of occurrence of the abnormal sensation, one or more posture types of the user body, duration of the abnormal sensation and past abnormal sensation history of the user body, and
wherein the one or more recommendations include measures to prevent the abnormal sensation in future and/or recommendations based on the one or more functions performed by the event modulation module.

3. The method of claim 1, wherein the detection module determines the level of abnormal sensation by:
classifying one or more movements of the user body in any one of one or more posture types of the user body, by a movement classification sub-module, wherein the one or more posture types of the user body include type A, type B, type C, and type D;
identifying changes in one or more bio-markers with respect to time, by biomarkers identification sub-module, wherein the one or more bio-markers include blood volume, blood flow rate, and heart rate, which are calculated from one or more key points identified on noiseless optical signal; and
determining the level of abnormal sensation in the user body, by an abnormal sensation detection sub-module, using the classified one or more posture types, identified changes in the one or more bio-markers, and external parameters associated with the abnormal sensation including at least but not limited to time duration of the one or more posture types.

4. The method of claim 3, wherein the movement classification sub-module performs classification of the one or more movements of the user body by:
extracting one or more features from predefined features, wherein the predefined features are features of the user body defined for devices including a gyroscope and an accelerometer for measuring one or more movements of the user body;
calculating values for the extracted one or more features from the predefined features; and
classifying the one or more movement in any one of the one or more posture types of the user body based on calculated values of extracted features for the respective one or more movement of the user body.

5. The method of claim 3, wherein the type A, type B, type C, and type D of the one or more posture types of the user body specifically for hand represents pressed hand posture, anti-gravity posture, user induced numbness, and vibrating hand syndrome respectively.

6. The method of claim 3, wherein the one or more key points on the noiseless optical signals are identified by:
receiving, from one or more sensors, a body reflected optical signal, wherein the one or more sensors include an optical sensor, a PPG sensor, and a camera sensor;
filtering noise from the received optical signal for generating a noiseless optical signal;
performing local maxima scalogram (LMS) matrix of the noiseless optical signal;
performing row wise summation of each and every LMS matrix;
removing all elements from original LMS matrix to perform LMS rescaling; and
performing peak detection analysis of the noiseless optical signal for identifying the one or more key points including starting point and a systolic peak, wherein the peak detection analysis includes performing column-wise standard deviation for finding one or more indices having standard deviation equals to one.

7. The method of claim 1, wherein the prevention module generates vibrations of required frequency by:
determining vibration of required frequency, by a vibration intensity determination sub-module, using the level of abnormal sensation in the user body and one or more health parameters which include age, ambient temperature, and diabetic status of the user body; and
generating vibrations of required frequency in the wearable device and the electronic device.

8. The method of claim 7, wherein the prevention module generates vibrations of required frequency in a localized region on display of the electronic device, using a vibration position identification sub-module, wherein the localized region is identified by computing length of swipe arc, made by touch of finger of the user body on the display of the electronic device, using length of the finger computed from positional co-ordinates of first touch point and last touch point on the display.

9. The method of claim 1, wherein the one or more functions performed by the event modulation module include:
identifying current window of the electronic device;
extracting list of one or more features of the electronic device in the identified current window;
identifying one or more features of the electronic device that degrades the user experience during the abnormal sensation;
determining available alternate one or more features of the electronic device; and
modulating the one or more features of the electronic device by disabling the identified one or more features of the electronic device that degrades the user experience and enabling the available alternate one or more features.

10. The method of claim 9, wherein the list of the one or more features of the electronic device includes at least but not limited to fingerprint authentication, face authentication, voice commands, iris authentication, text auto correction, haptic feedback for predictive text, customized keyboard, and auto size variation of keyboard.

11. A system for enhancing user experience of an electronic device during abnormal sensation in a user body, the system comprising:
a detection module configured to determine a level of abnormal sensation in the user body;
a prevention module, in communication with the detection module, configured to generate vibrations of required frequency in a wearable device and in the electronic device; and
an event modulation module in communication with the prevention module configured to enhance the user experience of the electronic device.

12. The system of claim 11,
wherein the system comprises: a recommendation module configured to provide one or more recommendations to a user, using an artificial intelligence based on frequency of occurrence of the abnormal sensation, one or more posture types of the user body, duration of the abnormal sensation and past abnormal sensation history of the user body, and
wherein the one or more recommendations include measures to prevent the abnormal sensation in future and/or recommendations based on one or more functions performed by the event modulation module.

13. The system of claim 11, wherein the detection module includes:
a movement classification sub-module configured to classify one or more movements of the user body in any one of one or more posture types of the user body, wherein the one or more posture types of the user body include type A, type B, type C, and type D;
a biomarkers identification sub-module configured to identify changes in one or more bio-markers with respect to time, wherein the one or more bio-markers include blood volume, blood flow rate, and heart rate, which are calculated from one or more key points identified on noiseless optical signal; and
an abnormal sensation detection sub-module configured to determine the level of abnormal sensation in the user body using the classified one or more posture types, identified changes in the one or more bio-markers and external parameters associated with the abnormal sensation including at least but not limited to time duration of the one or more posture types.

14. The system of claim 13, wherein the type A, type B, type C, and type D of the one or more posture types of the user body specifically for hand represents pressed hand posture, anti-gravity posture, user induced numbness, and vibrating hand syndrome respectively.

15. The system of claim 11, wherein the prevention module includes:
a vibration intensity determination sub-module configured to determine vibration of required frequency, wherein the vibration of required frequency is determined by using the level of abnormal sensation and one or more health parameters which include age, ambient temperature, and diabetic status of the user body and generating vibrations of required frequency in the wearable device and the electronic device; and
a vibration position identification sub-module configured to generate vibrations of required frequency in a localized region on display of the electronic device, using a vibration position identification sub-module, wherein the localized region is identified by computing length of swipe arc, made by touch of finger of the user body on the display of the electronic device, using length of the finger computed from positional coordinates of first touch point and last touch point on the display.

16. The system of claim 15, wherein a movement classification sub-module performs classification of one or more movements of the user body by:
extracting one or more features from predefined features, wherein the predefined features are features of the user body defined for devices including a gyroscope and an accelerometer for measuring one or more movements of the user body;
calculating values for the extracted one or more features from the predefined features; and
classifying the one or more movement in any one of one or more posture types of the user body based on calculated values of extracted features for the respective one or more movement of the user body.

17. The system of claim 15, wherein one or more key points on a noiseless optical signals are identified by:
receiving, from one or more sensors, a body reflected optical signal, wherein the one or more sensors include an optical sensor, a PPG sensor, and a camera sensor;
filtering noise from the received optical signal for generating a noiseless optical signal;
performing local maxima scalogram (LMS) matrix of the noiseless optical signal;
performing row wise summation of each and every LMS matrix;
removing all elements from original LMS matrix to perform LMS rescaling; and
performing peak detection analysis of the noiseless optical signal for identifying the one or more key points including starting point and a systolic peak, wherein the peak detection analysis includes performing column-wise standard deviation for finding one or more indices having standard deviation equals to one.

18. The system of claim 15, wherein one or more functions performed by the event modulation module include:
identifying current window of the electronic device;
extracting list of one or more features of the electronic device in the identified current window;
identifying one or more features of the electronic device that degrades the user experience during the abnormal sensation;
determining available alternate one or more features of the electronic device; and
modulating the one or more features of the electronic device by disabling the identified one or more features of the electronic device that degrades the user experience and enabling the available alternate one or more features.

19. The system of claim 18, wherein the list of the one or more features of the electronic device includes at least but not limited to fingerprint authentication, face authentication, voice commands, iris authentication, text auto correction, haptic feedback for predictive text, customized keyboard, and auto size variation of keyboard.

20. One or more non-transitory computer-readable storage media storing computer-executable instructions that, when executed by a processor individually or collectively, cause an electronic device to perform operations, the operations comprising
enhancing user experience of the electronic device during abnormal sensation in a user body;
determining, by a detection module, a level of abnormal sensation in the user body;
generating, by a prevention module, vibrations of required frequency in a wearable device and in the electronic device; and
performing, by an event modulation module, one or more functions for enhancing the user experience of the electronic device.
